SEQUENCE LISTING (6 sequences)

SEQ ID NO: 1 - protein, 59 aa, synthetic construct
  REGION 1..59: note = CALRmut
QDEEQRTRRMMRTKMRRMMRTKMRMRRMRRTRRKMRRKMSPARPRTSCREACLQGWTEA

SEQ ID NO: 2 - protein, 24 aa, synthetic construct
  REGION 1..24: note = 48250_1_w2
RRMMRTKMRMRRMRRTRRKMRRKM

SEQ ID NO: 3 - protein, 25 aa, synthetic construct
  REGION 1..25: note = 48250_2d
MRTKMRMRRMRRTRRKMRRKMSPAR

SEQ ID NO: 4 - protein, 25 aa, synthetic construct
  REGION 1..25: note = 48250_3
MRMRRMRRTRRKMRRKMSPARPRTS

SEQ ID NO: 5 - protein, 25 aa, synthetic construct
  REGION 1..25: note = 48250_4
RMRRTRRKMRRKMSPARPRTSCREA

SEQ ID NO: 6 - protein, 25 aa, synthetic construct
  REGION 1..25: note = 48250_6
MRRKMSPARPRTSCREACLQGWTEA

(19) United States
(12) Patent Application Publication
(10) Pub. No.: US 2025/0352627 A1
(43) Pub. Date: Nov. 20, 2025

(54) MUTANT CALR-PEPTIDE BASED VACCINE

(71) Applicant: Ichan School of Medicine at Mount Sinai, New York, NY (US)

(72) Inventors: Nina BHARDWAJ, New York, NY (US); Camelia IANCU-RUBIN, New York, NY (US); Ronald HOFFMAN, New York, NY (US); Cansu CIMEN BOZKUS, New York, NY (US)

(21) Appl. No.: 18/872,886

(22) PCT Filed: Jun. 15, 2023
(86) PCT No.: PCT/US2023/068539

§ 371 Date: Dec. 9, 2024

Related U.S. Application Data
(60) Provisional application No. 63/353,078, filed on Jun. 17, 2022.

(51) Int. Cl. A61K 39/00; A61K 39/39; A61P 37/04

(52) U.S. Cl. CPC A61K 39/0011; A61K 39/39; A61P 37/04; A61K 2039/55561; A61K 2039/6081; A61K 2039/70

(57) ABSTRACT

The presently claimed and described technology provides vaccine compositions comprising at least two mutant-calreticulin (CALR)-peptides, wherein the at least two peptides have overlapping sequences and methods for administration of the vaccine compositions to induce or elicit an antitumor response or improve or enhance antitumor T cell immunity and methods of preventing, treating, reducing, or slowing progression or development of a hematological malignancy in a subject with a calreticulin mutation.

### RELATED APPLICATIONS

The present patent application claims the priority benefit of U.S. Provisional Patent Application Ser. No. 63/353,078, filed Jun. 17, 2022, the content of which is hereby incorporated by reference in its entirety into this disclosure.

### BACKGROUND

Chronic myeloproliferative neoplasms (MPN) are a group of hematological malignancies with diverse clinical phenotypes. The Philadelphia chromosome negative MPN includes: Polycythemia Vera (PV), Essential Thrombocythemia (ET), and primary myelofibrosis (PMF). Furthermore, PV and ET can evolve to a clinical phenotype that resembles Myelofibrosis (MF) and each of these MPNs can progress to MPN-blast phase, which resembles acute myeloid leukemia but is almost universally refractory to intensive chemotherapy. MPN can be a debilitating and progressive disease. Many patients suffer from constitutional symptoms such as fatigue, night sweats, weight loss, pruritus, bone pain, and organomegaly and vasomotor symptoms that negatively affect their quality of life. Moreover, patients experience thrombotic and bleeding complications and can progress to an aggressive form of bone marrow failure or acute leukemia.

Current treatment options are limited, mainly supportive/symptomatic, and none can change the natural course of the disease. The only curative treatment for the MPNs is allogeneic stem cell transplantation. Still, it is limited in application by advanced patient age, comorbid conditions, lack of appropriate donor options, and often patient willingness. Treatment for ET is meant to reduce complications and control symptoms. It is based on estimation of the risk of complication (often using the International Prognostic Score of thrombosis in World Health Organization essential thrombocythemia (IPSET-thrombosis)) and includes aspirin and cytoreduction. Treatment for MF is most often based on risk stratification by various systems, including the Dynamic International Prognostic Scoring System (DIPSS), which allows a physician to a determine a treatment plan based on the specific clinical issues at hand (e.g., anemia, constitutional symptoms, symptomatic splenomegaly) considering the competing risks of treatment risk and disease risk. Ruxolitinib (Jakafi, Incyte) and Fedratinib (Inrebic, Celgene) are selective JAK½ oral tyrosine kinase inhibitors that are FDA-approved therapy for intermediate/high-risk MF patients with a relatively safe toxicity profile and proven efficacy in reducing splenomegaly and improving symptoms. However, they do not eliminate the malignant stem cells, and due to dose limiting thrombocytopenia, inability to alleviate anemia, loss of initial response, and failure to halt progression to acute leukemia, other therapies are desperately needed.

A number of driver gene mutations result in the activation of the JAK-STAT signaling pathway. This is a major event in disease initiation in MPNs. One such driver gene mutation occurs in the calreticulin gene (CALR), which is, after JAK2 (V617F) mutation, the second most common driver mutation in ET and MF patients (30%). CALR is a multifunctional protein that plays a critical role in the immune system by its involvement in immune-mediated phagocytic antigen uptake, T-cell activation, and major histocompatibility complex (MHC) assembly. Out of more than 50 types of mutations identified in the CALR gene, the vast majority (85%) consist of deletions (type I) and insertions (type II). Despite this heterogeneity, all these mutations cause a shift in the reading frame resulting in an identical 36-amino acid sequence in the C-terminus of the protein. This molecular defect confers a significant replicative advantage to transformed cells harboring CALR mutations by activating the JAK/STAT pathway through binding and activation of the thrombopoietin receptor.

Interestingly, MPN patients with CALR mutations have better overall survival (17.7 years) as compared to patients carrying JAK2 or MPL genes mutations (˜9.2 years) and to triple-negative patients (3.2 years). Unlike the wild-type protein, mutant-CALR lacks the KDEL sequence at the end of the C-terminus, which normally triggers retention of CALR in the endoplasmic reticulum. Exposure of CALR at the cell surface represents the major pro-phagocytic “eat-me” signal and initiates cancer cell clearance by specialized phagocytes, and is associated with enhanced antitumor immunity and better survival in AML patients. Due to the absence of KDEL sequence, it is possible that CALR mutations might lead to increased phagocytic uptake, increased antigen presentation, and induction of spontaneous immunity in these patients. In other words, this new epitope renders the mutant malignant cells more susceptible to immune-mediated clearance and T cell-mediated killing, which may be reflected, at least in part, by the superior survival observed in these patients.

### BRIEF SUMMARY

Current MPN treatments are geared toward symptom palliation and not toward changing the natural course of the disease. The mutated CALR neoantigen present in a patient with MPN represents an ideal antigen for targeted immunotherapy as it is stably and specifically expressed by the malignant cells and is absent in the normal tissues. CALR neoantigen is immunogenic, effector T cells are capable of recognizing this neo-antigen, and these specific effector T-cells can potently kill hematopoietic cells carrying the mutation in vitro. The inventors have discovered a peptide-based vaccine targeting mutated-CALR comprising overlapping long peptides spanning the last 44-aa of the C-terminal.

One aspect of the present disclosure is a vaccine composition comprising at least two mutant-calreticulin (CALR)-peptides, wherein the at least two peptides have overlapping sequences. In another aspect, the vaccine composition comprises at least three mutant-calreticulin (CALR)-peptides, alternatively at least four mutant-calreticulin (CALR)-peptides, alternatively at least five mutant-calreticulin (CALR)-peptides, wherein all peptides in the vaccine composition have overlapping sequences. In a further aspect, the overlapping sequences overlap by about 1 amino acid, alternatively by about 2 amino acids, alternatively by about 3 amino acids, alternatively by about 4 amino acids, alternatively by about 5 amino acids, alternatively by about 6 amino acids, alternatively by about 7 amino acids, alternatively by about 8 amino acids, alternatively by about 9 amino acids, alternatively by about 10 amino acids, alternatively by about 15 amino acids, alternatively by about 20 amino acids. In a further aspect, the at least two peptides comprise at least about 20 amino acids, alternatively at least about 22 amino acids, alternatively at least about 24 amino acids, alternatively at least about 25 amino acids, alternatively at least about 27 amino acids.

One aspect of the disclosure is a vaccine composition comprising at least two mutant-calreticulin (CALR)-peptides selected from the group consisting of SEQ ID NOs: 2, 3, 4, 5, and 6. In another aspect, the vaccine composition comprises at least three mutant-calreticulin (CALR)-peptides, alternatively at least four mutant-calreticulin (CALR)-peptides, alternatively at least five mutant-calreticulin (CALR)-peptides selected from the group consisting of SEQ ID NOs: 2, 3, 4, 5, and 6.

In an aspect, the vaccine composition further comprises a pharmaceutically acceptable carrier, an adjuvant, or helper peptide. In another aspect, the adjuvant is selected from the group consisting of Polyinosinic-Polycytidylic Acid stabilized with Polylysine and Carboxymethylcellulose (Poly-ICLC), Keyhole limpet hemocyanin (KLH), and combinations thereof. In yet another aspect, the vaccine composition is configured to be administered intramuscularly, intranodally, or subcutaneously.

In an aspect, the vaccine composition induces or elicits an antitumor response or improves or enhances antitumor T cell immunity in a subject in need thereof. In another aspect, the antitumor response is a CD4 and/or CD8 T cell response. In yet another aspect, the subject is a human suffering from a hematological malignancy with a calreticulin mutation. In a further aspect, the hematological malignancy is a myeloproliferative neoplasm.

One aspect of the disclosure is a method of inducing or eliciting an antitumor response or improving or enhancing antitumor T cell immunity in a subject in need thereof, the method comprising administering an effective amount of a disclosed vaccine composition.

In another aspect, the subject is a human suffering from a hematological malignancy with a calreticulin mutation. In yet another aspect, the hematological malignancy is a myeloproliferative neoplasm.

One aspect of the disclosure is a method of preventing, treating, reducing, or slowing progression or development of a hematological malignancy with a calreticulin mutation in a subject in need thereof, the method comprising administering an effective amount of a disclosed vaccine composition. In another aspect, the hematological malignancy is a myeloproliferative neoplasm.

One aspect of the disclosure is a method of preventing, reducing, or slowing progression or development of a hematological malignancy with a calreticulin mutation in a subject at risk of developing a hematological malignancy with a calreticulin mutation, the method comprising determining if a subject is at risk of developing a hematological malignancy with a calreticulin mutation and administering to the subject an effective amount of a disclosed vaccine composition.

In another aspect, determining if a subject is at risk of developing a hematological malignancy with a calreticulin mutation comprises detecting a calreticulin mutation in a sample from the subject. In a further aspect, the sample is a biological sample. In yet a further aspect, the biological sample is selected from the group consisting of blood, tissue, cells, urine, saliva, and biological fluids. In another aspect, the hematological malignancy is a myeloproliferative neoplasm.

These and other advantages, aspects, and novel features of the present disclosure, as well as details of an illustrated embodiment thereof, will be more fully understood from the following description and drawings.

### BRIEF DESCRIPTION OF THE DRAWINGS

Various aspects of the present disclosure will now be described, by way of example only, with reference to the attached Figures, wherein:

FIG. 1 illustrates a comparison of mutant CALR peptides compared to wild-type (WT) CALR peptides.

FIGS. 2A-2F depict T cell immune response evaluation after stimulation with mutant-CALR peptides in healthy donors. Peripheral blood mononuclear cells (PBMCs) were stimulated in vitro with pooled overlapping peptides spanning the C-terminal region of mutated CALR or the corresponding WT sequence. (FIG. 2A) Representative Enzyme-Linked Immunosorbent Spot (ELISPOT) data shows an increase in spot numbers upon priming with mutant-CALR peptide pool. CEFT stimulation was used as peptide-specific positive control. (FIG. 2B) Summary of ELISPOT data (n=16). Each data point represents one healthy donor. (FIG. 2C) Percentage of CD137*IFNγ⁺ T cells. Fold change=Number of spots for peptide/Number of spots for MOG peptide which was used as a negative control. IFNγ production is determined by intracellular staining and flow cytometry. (FIG. 2D) Representative flow plot showing that both CD4⁺ and CD8⁺ T cells produced IFNγ upon priming with mutant-CAL peptides pool. (FIG. 2E) Summary of IFNγ production by T cells (n=15). (FIG. 2F) Mutant CALR specific T cells were originated from the native pool (CD45RA+CCR7⁺CD45RO⁻), but not the memory pool (CD45RA; CD45RO⁺).

FIGS. 3A-3D depict T cell immunity against mutant-CALR in MPN patients. PBMCs from CALR+MPN patients were expanded in vitro following stimulation with WT or mutant-CALR OLPs. Stimulation with CEFT pool was used as a control. Expanded T cells were re-stimulated with either the peptide pool they were expanded with or the control peptide pool MOG. Representative ELISPOT images (FIG. 3A) and a summary of ELISPOT results (FIG. 3B) were generated in PBMCs from 18 CALR+MPN patients. Each data point represents one MPN patient. Statistical significance was evaluated by Wilcoxon signed-rank test: *, p=0.0327. Representative flow cytometry plots (FIG. 3C) and summary of intracellular staining analysis for IFN-γ in CD4 and CD8 T cell subsets of 11 CALR+MPN patients (FIG. 3D). Statistical significance for MOG vs. mutant-CALR OLPs was evaluated by Wilcoxon signed-rank test. P values were 0.0113 and 0.3223 for CD4⁺ and CD8⁺ T cells, respectively. The spot numbers and % IFN-γ values were calculated by subtracting the values obtained after MOG stimulation from the values after OLP pool stimulation, and negative values were set to zero. Horizontal lines indicate the mean.

FIGS. 4A and 4B depict T cells from MPN patients are exhausted and blockade of checkpoint receptors restore mutant-CALR-specific T cell immunity in vitro. PBMCs from CALR+MPN patients were stimulated in vitro with pooled mutant-CALR in the absence or presence of monoclonal antibodies blocking PD-1 or CTLA-4 (10 μg/mL). Representative IFN-γ ELISPOT images (FIG. 4A) and a summary of ELISPOT results (FIG. 4B) were generated in PBMCs from 18 CALR+MPN patients. Each data point represents one MPN patient. The change in spot numbers were displayed as fold change by dividing the number of spots formed after OLP pool stimulation to the number of spots formed after MOG stimulation. Horizontal lines indicate the median. Statistical significance for changes at population level was evaluated by Wilcoxon signed rank test. Isotype vs a-PD-1: p=0.3465, isotype vs CTLA-4:0.4171. Additionally, statistical significance was evaluated for each subject by t test by comparing isotype vs. checkpoint blockade. Three subjects that showed a significant response to checkpoint blockade were denoted. *p=0.0121, ** p=0.0045, *** p=0.0005.

FIG. 5 depicts exemplary immunogenicity assays with synthesized overlapping long peptides.

FIG. 6 depicts a method of administration of a vaccine composition according to an aspect of this disclosure.

### DETAILED DESCRIPTION

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

The invention is defined in the claims. However, below is a non-exhaustive listing of non-limiting exemplary aspects. Any one or more of the features of these aspects may be combined with any one or more features of another example, embodiment, or aspect described herein.

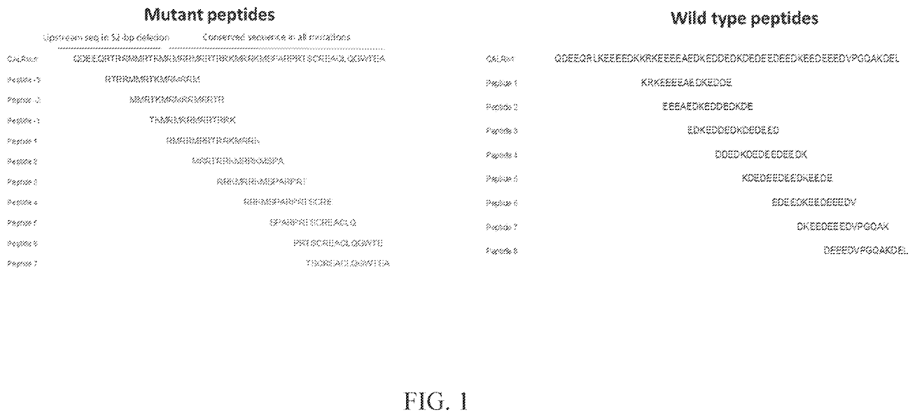

Somatic mutations in the calreticulin (CALR) gene are key drivers of cellular transformation in myeloproliferative neoplasms (MPN) and are the second most common driver mutations in essential thrombocythemia (ET) and myelofibrosis (MF) patients. All CALR mutations identified to date in MPN patients lead to a +1 shift in the open reading frame and result in the formation of an altered C-terminal, where the last 36-amino acid (aa) and 44-aa sequence are shared in 100% and >99% of all CALR+MPN patients, respectively. FIG. 1 illustrates a comparison of mutant CALR peptides compared to wild-type peptides. The recurrence and uniformity of the altered protein mark the mutated-CALR an attractive candidate as an MPN-specific tumor neoantigen that might elicit antitumor immune responses across patients who express this mutation. This altered protein results in a MPN-specific shared neo-antigen which has been shown to elicit immune responses in vitro and is a target of spontaneous T cell responses in vivo. As neoantigen-specific T cells are not subject to immune tolerance, they have the potential to exhibit strong effector responses, specifically against malignant cells that express the neoantigen.

Epitopes within the altered CALR C-terminus can elicit specific CD4 and CD8 T cell responses, with no cross-reactivity to WT protein expressed on nonmalignant cells, in a subset of CALR+MPN patients. These findings establish the mutated-CALR as an MPN-specific neoantigen that can be targeted by various immunotherapy approaches, including neoantigen-specific vaccines and adoptive T cell therapies for the elimination of malignant clones in CALR+MPN patients.

In an aspect, the disclosure provides a vaccine composition comprising at least two mutant-calreticulin (CALR)-peptides, wherein the at least two peptides have overlapping sequences. One of ordinary skill can appreciate that the vaccine composition may comprise several overlapping peptides. In a non-limiting example, the vaccine composition may comprise at least three mutant-calreticulin (CALR)-peptides, alternatively at least four mutant-calreticulin (CALR)-peptides, or alternatively at least five mutant-calreticulin (CALR)-peptides, wherein all peptides in the vaccine composition have overlapping sequences.

The overlapping peptides may overlap by at least one amino acid; however, depending on the mutant-calreticulin (CALR)-peptides in the vaccine composition, the overlapping sequences may overlap by about 2 amino acids, alternatively by about 3 amino acids, alternatively by about 4 amino acids, alternatively by about 5 amino acids, alternatively by about 6 amino acids, alternatively by about 7 amino acids, alternatively by about 8 amino acids, alternatively by about 9 amino acids, alternatively by about 10 amino acids, alternatively by about 15 amino acids, alternatively by about 20 amino acids.

The vaccine composition may also comprise long peptides. “Long peptides” or “synthetic long peptides” include peptides that are at least about 15 amino acids in length. In some aspects, the vaccine composition comprises at least two mutant-calreticulin (CALR)-peptides, wherein each mutant-calreticulin (CALR)-peptide is at least about 20 amino acids in length, alternatively at least about 22 amino acids in length, alternatively at least about 24 amino acids in length, alternatively at least about 25 amino acids in length, alternatively at least about 27 amino acids in length.

Peptides that are 8-11 amino acids long are presented on MHC class I molecules and are recognized by CD8⁺ T cells, which then mediate the cytotoxic response to these types of cellular antigens. Therapeutic vaccines targeting tumor antigens using minimal peptide epitopes (8-11mer) may lead to detectable epitope-specific CD8⁺ cytotoxic T cell (CTL) responses in vitro and in vivo, which may in turn inhibit tumor growth in vivo. However, vaccines based on minimal peptide epitopes often suffer from a lack of consistent CD8⁺ T-cell induction due to the fact that a minimal epitope may bind directly to MHC class I molecules on the cell surface, leading to an inefficient presentation of antigens by professional antigen-presenting cells (pAPC), or presentation of the epitope by non-professional APC that lack the necessary signals for robust CTL activation. Inefficient pMHC presentation induced by minimal peptide epitope vaccine platforms may induce CTL energy and/or specific tolerance towards tumor antigens, which may in turn facilitate tumor outgrowth.

Vaccination platforms using synthetic long peptides (SLP) are superior to those utilizing minimal peptide epitopes in many respects. SLPs, generally 15-35mers, are not able to bind directly to class I MHC. They are thus more likely to be processed by dendritic cells and other pAPC and more likely to be presented in the draining lymph node in the presence of the appropriate cytokine milieu and co-stimulatory signals. This route of vaccination appears to result in the production of a superior epitope-specific CTL response, as determined by markers of cytotoxicity or tumor-lytic activity. Where vaccination with minimal epitopes frequently leads to a transient response, vaccination with long peptides appears to promote durable memory responses, which may be attributable to the generation of class II restricted epitopes, resulting in the activation of epitope-specific CD4⁺ T helper lymphocytes.

Endogenous antigen-specific CD8⁺CTL is rarely present at baseline but is consistently induced by SLP-based vaccine platforms, particularly in subjects who remained on protocol long enough to receive the full vaccination course. This suggests that SLP-based vaccines may enhance or induce antigen-specific CD8⁺CTL responses in vivo as determined by IFNγ ELISPOT and multi-parameter flow cytometry. SLP vaccines also consistently induce circulating CD4⁺ T cell responses, which may be attributable to efficient processing and presentation of the SLP by pAPC. Vaccination with SLP frequently induces long-term immunologic memory responses in patients following multiple courses of vaccination, which may be detectable up to twenty-four months after vaccination without additional maintenance dosing.

Sequences of the CALRMUT and exemplary mutant-CALR peptides of the present disclosure are shown in Table 1.

TABLE 1
Amino Acid Sequence of CALR_{MUT} and exemplary mutant-CALR peptides

| Internal Identifier | SEQ ID NO | Sequence |
|---|---|---|
| CALR_{MUT} | SEQ ID NO: 1 | QDEEQRTRRMMRTKMRRMMRTKMRMRRMRR |
| | | TRRKMRRKMSPARPRTSCREACLQGWTEA |
| 48250_1_w2 | SEQ ID NO: 2 | RRMMRTKMRMRRMRRTRRKMRRKM |
| 48250_2d | SEQ ID NO: 3 | MRTKMRMRRMRRTRRKMRRKMSPAR |
| 48250_3 | SEQ ID NO: 4 | MRMRRMRRTRRKMRRKMSPARPRTS |
| 48250_4 | SEQ ID NO: 5 | RMRRTRRKMRRKMSPARPRTSCREA |
| 48250_6 | SEQ ID NO: 6 | MRRKMSPARPRTSCREACLQGWTEA |

In an aspect, the disclosure provides for vaccine compositions comprising at least two of the exemplary mutant-CALR peptides. In a non-limiting example, the vaccine composition may comprise at least two exemplary mutant-CALR peptides selected from SEQ ID NO: 2, SEQ ID NO: 3, SEQ ID NO:4, SEQ ID NO:5, or SEQ ID NO:6. It should further be appreciated that the vaccine composition may comprise at least three mutant-calreticulin (CALR)-peptides, alternatively at least four mutant-calreticulin (CALR)-peptides, or alternatively at least five mutant-calreticulin (CALR)-peptides selected from SEQ ID NO: 2, SEQ ID NO:3, SEQ ID NO:4, SEQ ID NO:5, or SEQ ID NO:6. In a non-limiting example, the vaccine composition comprises SEQ ID NO: 2, SEQ ID NO:3, SEQ ID NO:4, SEQ ID NO:5, or SEQ ID NO:6. As shown in Table 1, all five peptides comprise the same overlapping region of MRRKM.

Previous vaccine compositions comprised a single peptide, covering the 36 aa of mutated CALR C terminus, with montanide as an adjuvant and used in a mixed population of MPN patients, including ET and MF patients. This is contrary to the disclosed vaccine compositions that comprise several overlapping long peptides, each around 25 amino acids, spanning the 44 amino acids of mutated CALR C terminus. The use of overlapping peptides unexpectantly increases the efficiency of antigen presentation, yields a greater amount of immunogenic epitopes, and improves the antitumor T cell immunity the vaccine will elicit. Furthermore, targeting the 44 aa of mutated protein, instead of 36 aa, increases the breadth of T cell responses elicited after immunization by providing additional neoepitopes. Accordingly, previous findings demonstrated that the mutated CALR sequences upstream of the 36 aa C terminus, included within the 44 aa tail, generate immunogenic neoepitopes (Cimen Bozkus et al., 2019).

Instead of montanide as an adjuvant, some aspects of the described vaccine compositions comprise Polyinosinic-Polycytidylic Acid stabilized with Polylysine and Carboxymethylcellulose (Poly-ICLC) or Keyhole Limpet Hemocyanin (KLH) as adjuvants. Vaccine adjuvants are compounds used to increase the immunogenicity of a given antigen. They serve to enhance the magnitude, breadth, quality, and longevity of specific immune responses to antigens but have minimal toxicity or lasting immune effects on their own. Effective adjuvants function to activate the innate immune system, such as through TLR signaling.

In some aspects, the described vaccine compositions comprise both Poly-ICLC and KLH. As a therapeutic viral-mimic, Poly-ICLC alone has immune enhancing properties. Similarly, KLH can act as an immunostimulatory molecule in addition to its role as a protein carrier. Overall, KLH has been shown to boost the CD4⁺ T cell mediated helper immune response that serves to amplify the vaccine-induced CD8⁺ T cell responses.

The vaccine compositions may also be administered using any suitable route of administration including, but not limited to, an intravenous route (IV), an intramuscular route (IM), a subcutaneous route (Subcut), or an intranodal route. IV injections are administered into a vein and directly into the bloodstream. IM injections are administered into the muscle through the skin and subcutaneous tissue. Subcut injections are administered into the fatty tissue found below the dermis and above muscle tissue.

In some aspects, the disclosed vaccine compositions induce or elicit an antitumor response or improve or enhance antitumor T cell immunity in a subject in need thereof. In a non-limiting aspect, the antitumor response is a CD4 and/or CD8 T cell response, and the subject is a human suffering from a hematological malignancy with a calreticulin mutation, such as a myeloproliferative neoplasm.

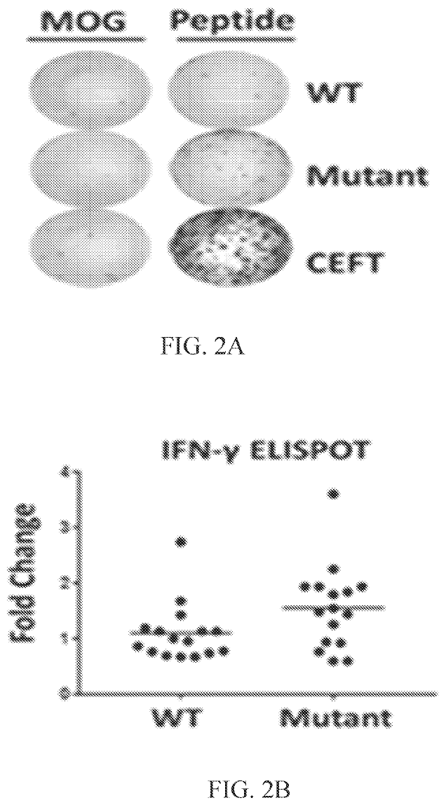

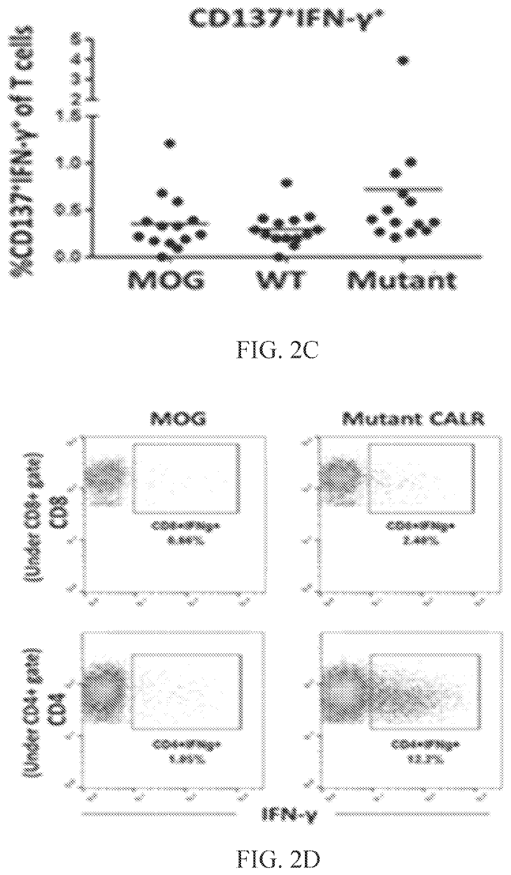

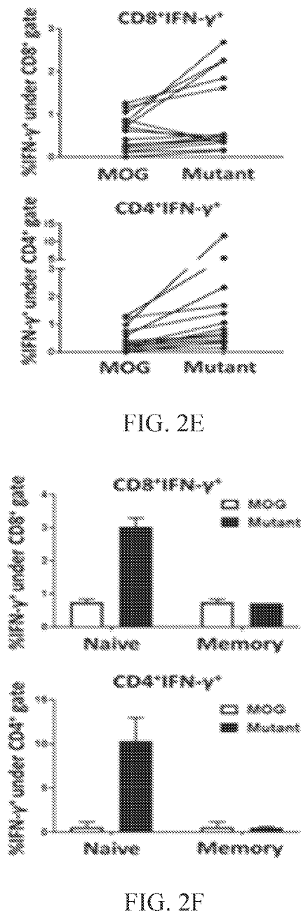

The immunogenicity of mutant-CALR peptides spanning the mutated CALR region in vitro was evaluated by utilizing intracellular staining (ICS) and Enzyme-Linked ImmunoSPOT (ELISPOT) assays. Naïve T cells derived from the peripheral blood mononuclear cells (PBMCs) of healthy donors displayed effector functions after priming with the mutated peptides but not with the corresponding wild-type (WT) peptides (FIGS. 2A and 2B). Incubation with the mutant-CALR peptides induced T cell proliferation, upregulation of CD137, and production of interferon (IFN)-γ (FIG. 2C). There was a significant increase in IFN-γ production by CD4″ T cells and a 2-3 fold increase by CD8⁺ T cells after stimulation with mutated peptides compared to control self-antigens (FIGS. 2D-2F). These observations indicate the immunogenicity of mutant-CALR neoantigen.

In some aspects, the disclosed vaccine compositions are administered to a subject in various methods and/or routes of administration. The term “subject” may be used interchangeably with the term “patient.” Additionally, the subject or patient may be a mammal, for example, a human. Non-limiting examples of methods of this disclosure include methods of inducing or eliciting an antitumor response or improving or enhancing antitumor T cell immunity in a subject, preventing, treating, reducing, or slowing the progression or development of a hematological malignancy with a calreticulin mutation in a subject, and/or method of preventing, reducing, or slowing progression or development of a hematological malignancy with a calreticulin mutation in a subject at risk of developing a hematological malignancy with a calreticulin mutation, the method comprising determining if a subject is at risk of developing a hematological malignancy with a calreticulin mutation. In the disclosed methods, the subject is, for example, a human suffering from a hematological malignancy with a calreticulin mutation wherein the hematological malignancy is a myeloproliferative neoplasm.

In an aspect, determining if a subject is at risk of developing a hematological malignancy with a calreticulin mutation comprises detecting a calreticulin mutation in a sample from the subject. In non-limiting examples, the sample is a biological sample, such as blood, tissue, cells, urine, saliva, and/or biological fluids. Additionally, if a subject is deemed to be at risk of developing a hematological malignancy with a calreticulin mutation, the subject may be administered an effective amount of the disclosed vaccine compositions.

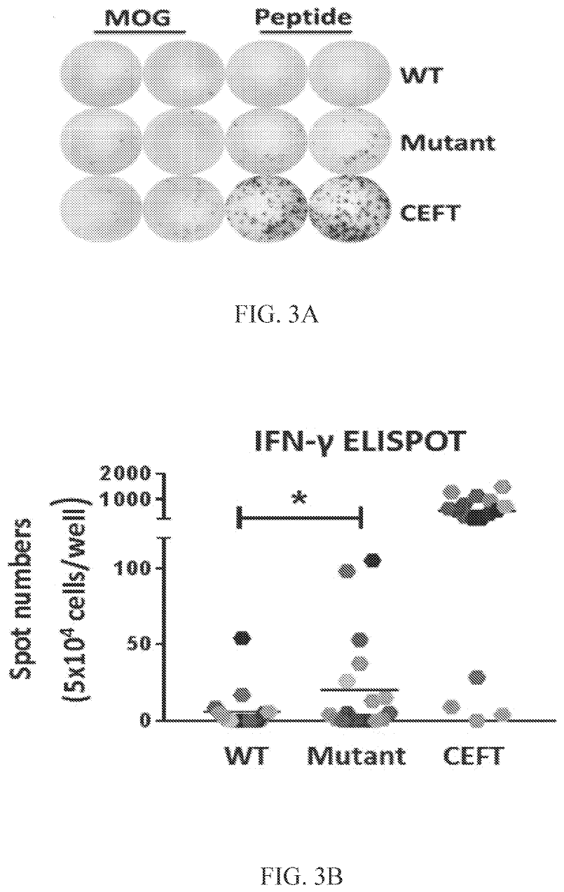

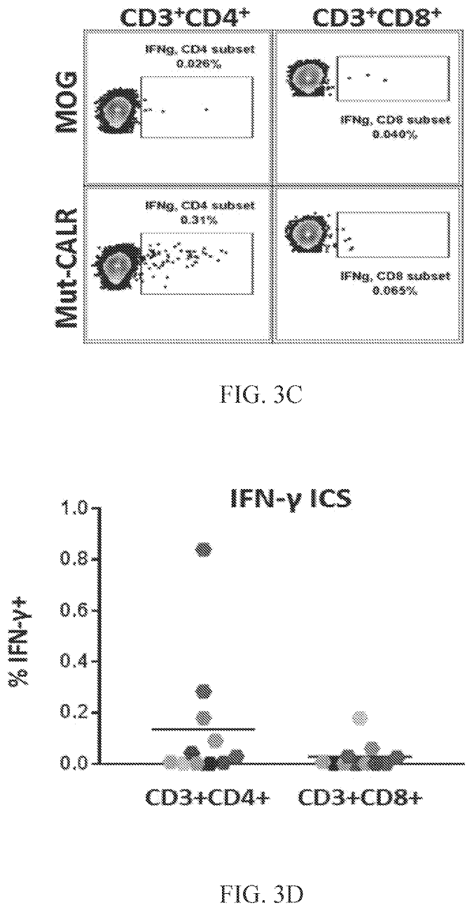

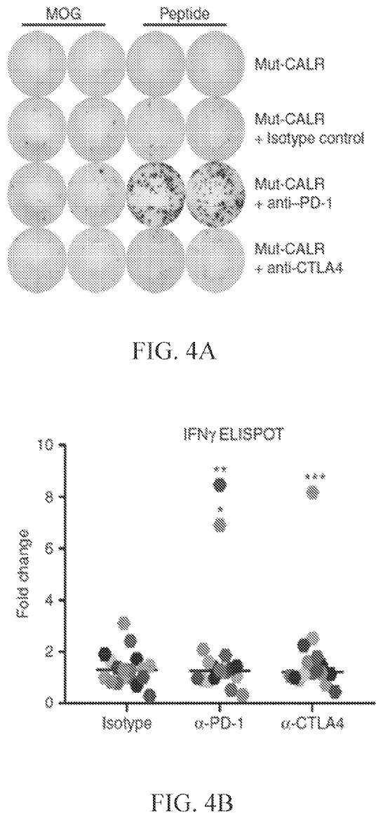

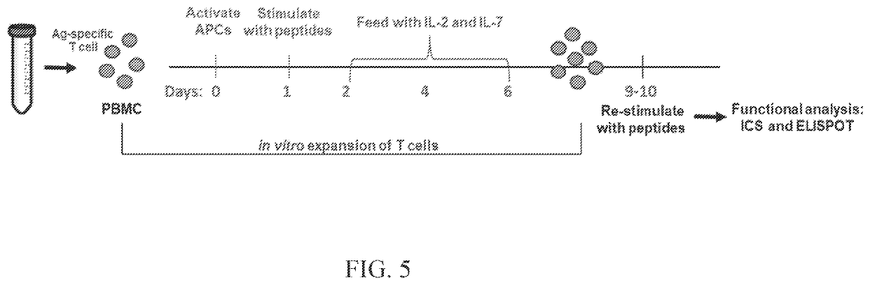

Immunogenicity assays were performed to evaluate mutant-CALR-specific T cell responses in PBMCs from MPN patients carrying CALR mutations. FIG. 5 is an exemplary depiction of immunogenicity assays. Eighteen patients with CALR+ET (n=7), post ET MF (n=7), or primary MF (n=4) were assessed. A subset of patients exhibited a significant increase in IFNγ production when the cells were stimulated with mutant-CALR overlapping peptides (OLPs) as compared with WT OLPs that spanned the C-terminus tail of the protein (FIGS. 3A and 3B). Notably, mutant-CALR-induced IFNγ production was observed with a greater frequency in ET, but not in patients with primary MF. Mutant-CALR-induced IFNγ production was observed primarily in CD4⁺ T cells (FIGS. 3C and 3D).

In vivo T cell priming in CALR+MPN patients was also assessed. For this, ex vivo T cell ELISPOT assays using PBMCs from a total of 19 JAK2V617F⁺ and 22 CALR MPN patients were performed. PBMCs from MPN patients were stimulated with mutant-CALR OLPs or control peptides, and mut-CALR-specific T cell responses were monitored after 48 hours. No mutant-CALR-specific T cell responses were detected ex vivo. This less robust mutant-CALR specific T cell response was attributed to potential immune inhibitory mechanisms present in MPN patients and the development of an exhaustion state due to chronic antigen exposure. Indeed, it was found that T cells from MPN patients exhibited higher expression of multiple cell-surface inhibitory molecules than healthy donor T cells. These were mainly checkpoint receptors, PD-1 and CTLA4 found on both CD8⁺ and CD4⁺ T-cell subsets. To confirm, mutant-CALR-specific T-cell responses in CALR+MPN PBMCs in the context of PD-1 or CTLA4 blockade were re-examined and it was found that T-cell responses against mutant-CALR OLPs were recovered in three CALR+MPNs patients (FIGS. 4A and 4B).

The peptide-based mutated-CALR vaccine composition and the identification of mutated-CALR-specific TCRs will unlock new treatment options for CALR+MPN patients. Mutated-CALR vaccination will induce antitumor T cell immunity, and as mutated CALR is a driver of MPN transformation, vaccine-induced T cells will eliminate disease-causing malignant cells. Additionally, administration of the disclosed vaccine compositions to CALR+ET patients will likely result in the development of greater magnitude and frequency of antitumor T cell responses, enabling the discovery of the features of anti-mutated CALR T cells in downstream applications. For example, autologous or HLA-matched healthy donor T cells that will be genetically modified to express mutated-CALR-specific TCRs and then adoptively transferred to CALR+MPN patients. Given that the disease is not curable, it is essential to identify new approaches that advance the care of these patients.

### Examples

### Mutated Calreticulin Vaccination Preparation

A dose of Mut-CALR vaccine consists of six (6) synthetic long peptides-200 μg (0.02 mL, 10 mg/mL) per peptide, KLH-100 μg (0.01 mL,10 ug/uL) (First vaccine only), PolyICLC-1.4 mg (0.78 mL, 1.8 mg/mL) and Normal saline: 0.085 mL (for the first vaccine only) or 0.09 mL.

A dose is prepared as two (2) separate, individually labeled mixtures, entitled: Mixture A and Mixture B. Each mixture consists of a pool of three (3) synthetic peptides (200 μg; 0.02 mL at 10 mg/mL per peptide), KLH antigen-first vaccine only (50 μg; 0.005 mL at 10 ug/uL) and PolyICLC (0.7 mg, 0.390 mL at 1.8 mg/mL). Finally, sterile normal saline (0.085 mL for the first vaccine and 0.09 mL for the rest). Each Mixture has a final volume of 0.500 mL. Mixtures A and B will be drawn into separate 2 mL syringes and prepared for administration.

TABLE 2
Preparing the “first” vaccine with KLH

| | Mixture A | | Mixture B | |
|---|---|---|---|---|
| Mutant-CALR-peptides | 0.02 ml × 3 = 0.06 ml | | 0.02 ml × 3 = 0.06 ml | |
| (200 ug of each of the 6) | | | | |
| KLH (100 ug) | 0.005 | ml | 0.005 | ml |
| PolyICLC (1.4 mg) | 0.390 | ml | 0.390 | ml |
| Sterile saline | 0.045 | ml | 0.045 | ml |
| TOTAL VOLUME | 0.5 | ml | 0.5 | ml |

TABLE 3
Preparing Mutant-CALR vaccine without KLH

| | Mixture A | | Mixture B | |
|---|---|---|---|---|
| Mutant-CALR-peptides | 0.02 ml × 3 = 0.06 ml | | 0.02 ml × 3 = 0.06 ml | |
| (200 ug of each of the 6) | | | | |
| KLH (100 ug) | N/A | | N/A | |
| PolyICLC (1.4 mg) | 0.390 | ml | 0.390 | ml |
| Sterile saline | 0.05 | ml | 0.05 | ml |
| TOTAL VOLUME | 0.5 | ml | 0.5 | ml |

Each vaccine containing Poly-ICLC must be administered within two (2) hours of formulation.

### Mutated Calreticulin Vaccination Study Schema

### Inclusion Criteria

The selected subjects are ≥18 years of age and have a confirmed diagnosis of chronic phase MPN: high risk ET (HU failure/intolerance), low-intermediate 1 (DIPSS 0-1) PMF. The subjects also presented a verified mutation in CALR exon 9 and have adequate organ function.

### Treatment Plan

Each subject received ten (10) doses of Mutant-CALR peptides with KLH as a helper peptide (in the first vaccine only). The mutant-CALR vaccine was then administered every 2 weeks for the first 4 doses and then every 4 weeks for additional 6 doses on weeks 1, 3, 5, 7, 11, 15, 19, 23, 27 and 31.

Each subject also received ten (10) doses of Poly-ICLC. Poly-ICLC on weeks 1, 3, 5, 7, 11, 15, 19, 23, 27 and 31. Each Poly-ICLC dose was given the day after the corresponding Mut-CALR vaccination.

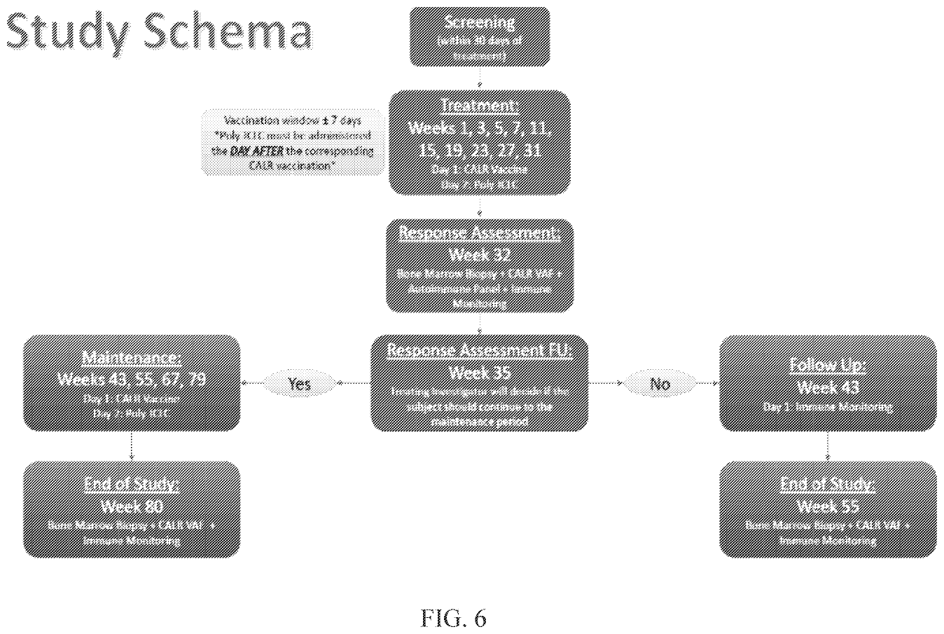

Subjects that maintained stable disease or showed disease improvement and didn't develop significant TRAE during the course of the vaccine treatment were entered into a maintenance treatment plan where they received up to four (4) additional Mutant-CALR vaccine and four (4) Poly-ICLC administrations, 12 weeks apart. During the maintenance phase, the Mutant-CALR vaccine was administered on Wk43, Wk55, Wk67, and Wk79, and the Poly-ICLC was administered the day after each corresponding Mutant-CALR vaccine. A detailed illustration of an exemplary mutated calreticulin vaccination study schema is illustrated in FIG. 6.

### REFERENCES

Cimen Bozkus, C. et al. (2019) “Immune Checkpoint Blockade Enhances Shared Neoantigen-Induced T-cell Immunity Directed against Mutated Calreticulin in Myeloproliferative Neoplasms,” Cancer discovery, 9 (9), pp. 1192-1207. doi: 10.1158/2159-8290.CD-18-1356.
Cimen Bozkus, C. et al. (2021) “A T-cell-based immunogenicity protocol for evaluating human antigen-specific responses,” STAR protocols, 2 (3), p. 100758. doi: 10.1016/j.xpro.2021.100758.
Ha, J.-S. and Kim, Y.-K. (2015) “Calreticulin exon 9 mutations in myeloproliferative neoplasms,” Annals of laboratory medicine, 35 (1), pp. 22-27. doi: 10.3343/alm.2015.35.1.22.
Handlos Grauslund, J. et al. (2021) “Therapeutic Cancer Vaccination With a Peptide Derived From the Calreticulin Exon 9 Mutations Induces Strong Cellular Immune Responses in Patients With CALR-Mutant Chronic Myeloproliferative Neoplasms,” Frontiers in oncology, 11, p. 637420. doi: 10.3389/fonc.2021.637420.
Hobbs, G. et al. (2021) “PD-1 inhibition in advanced myeloproliferative neoplasms,” Blood advances. American Society of Hematology, 5 (23), pp. 5086-5097. doi: 10.1182/bloodadvances.2021005491.
Holmström, M. O. et al. (2016) “The CALR exon 9 mutations are shared neoantigens in patients with CALR mutant chronic myeloproliferative neoplasms,” Leukemia, 30 (12), pp. 2413-2416. doi: 10.1038/leu.2016.233.
Holmström, M. O. et al. (2018) “The calreticulin (CALR) exon 9 mutations are promising targets for cancer immune therapy,” Leukemia, 32 (2), pp. 429-437. doi: 10.1038/leu.2017.214.
Keskin, D. B. et al. (2019) “Neoantigen vaccine generates intratumoral T cell responses in phase Ib glioblastoma trial,” Nature, 565 (7738), pp. 234-239. doi: 10.1038/s41586-018-0792-9.
Klampfl, T. et al. (2013) “Somatic mutations of calreticulin in myeloproliferative neoplasms,” The New England journal of medicine, 369 (25), pp. 2379-2390. doi: 10.1056/NEJMoa1311347.
Mascarenhas, J. and Hoffman, R. (2013) “A comprehensive review and analysis of the effect of ruxolitinib therapy on the survival of patients with myelofibrosis,” Blood, 121 (24), pp. 4832-4837. doi: 10.1182/blood-2013-02-482232.
Nangalia, J. et al. (2013) “Somatic CALR mutations in myeloproliferative neoplasms with nonmutated JAK2,” The New England journal of medicine, 369 (25), pp. 2391-2405. doi: 10.1056/NEJMoa1312542.
Ott, P. A. et al. (2017) “An immunogenic personal neoantigen vaccine for patients with melanoma,” Nature, 547 (7662), pp. 217-221. doi: 10.1038/nature22991.
Tubb, V. M. et al. (2018) “Isolation of T cell receptors targeting recurrent neoantigens in hematological malignancies,” Journal for immunotherapy of cancer, 6 (1), p. 70. doi: 10.1186/s40425-018-0386-y.
Zhao, L. and Cao, Y. J. (2019) “Engineered T Cell Therapy for Cancer in the Clinic,” Frontiers in immunology, 10, p. 2250. doi: 10.3389/fimmu.2019.02250.
Passamonti F, Cervantes F, Vannucchi A M, et al. A dynamic prognostic model to predict survival in primary myelofibrosis: a study by the IWG-MRT (International Working Group for Myeloproliferative Neoplasms Research and Treatment). Blood 2010; 115 (9): 1703-8.
Harrison C, Kiladjian J J, Al-Ali H K, et al. JAK inhibition with ruxolitinib versus best available therapy for myelofibrosis. N Engl J Med 2012; 366 (9): 787-98.
Verstovsek S, Mesa R A, Gotlib J, et al. A double-blind, placebo-controlled trial of ruxolitinib for myelofibrosis. N Engl J Med 2012; 366 (9): 799-807.
Pardanani A, Harrison C, Cortes J E, et al. Safety and Efficacy of Fedratinib in Patients With Primary or Secondary Myelofibrosis: A Randomized Clinical Trial. JAMA Oncol 2015; 1 (5): 643-51.
Chachoua I, Pecquet C, El-Khoury M, et al. Thrombopoietin receptor activation by myeloproliferative neoplasm associated calreticulin mutants. Blood 2016; 127 (10): 1325-35.
Cazzola M, Kralovics R. From Janus kinase 2 to calreticulin: the clinically relevant genomic landscape of myeloproliferative neoplasms. Blood 2014; 123 (24): 3714-9.
Rumi E, Pietra D, Pascutto C, et al. Clinical effect of driver mutations of JAK2, CALR, or MPL in primary myelofibrosis. Blood 2014; 124 (7): 1062-9.
Chao M P, Alizadeh A A, Tang C, et al. Anti-CD47 antibody synergizes with rituximab to promote phagocytosis and eradicate non-Hodgkin lymphoma. Cell 2010; 142 (5). 699-713.
Fucikova J, Truxova I, Hensler M, et al. Calreticulin exposure by malignant blasts correlates with robust anticancer immunity and improved clinical outcome in AML patients. Blood 2016; 128 (26): 3113-24.
Holmstrom M O, Riley C H, Skov V, Svane I M, Hasselbalch HC, Andersen MH. Spontaneous T-cell responses against the immune check point programmed-death-ligand 1 (PD-L1) in patients with chronic myeloproliferative neoplasms correlate with disease stage and clinical response. Oncoimmunology 2018; 7 (6): e1433521.
Foley E J. Antigenic properties of methylcholanthrene-induced tumors in mice of the strain of origin. Cancer Res 1953; 13 (12): 835-7.
Prehn R T, Main J M. Immunity to methylcholanthrene-induced sarcomas. J Natl Cancer Inst 1957; 18 (6): 769-78.
Klein G, Sjogren H O, Klein E, Hellstrom K E. Demonstration of resistance against methylcholanthrene-induced sarcomas in the primary autochthonous host. Cancer Res 1960; 20:1561-72.
Kripke M L. Antigenicity of murine skin tumors induced by ultraviolet light. J Natl Cancer Inst 1974; 53 (5): 1333-6.
Wortzel R D, Philipps C, Schreiber H. Multiple tumour-specific antigens expressed on a single tumour cell. Nature 1983; 304 (5922): 165-7.
Vansteenkiste J, De Ruysscher D, Eberhardt W E, et al. Early and locally advanced non-small-cell lung cancer (NSCLC): ESMO Clinical Practice Guidelines for diagnosis, treatment and follow-up. Ann Oncol 2013; 24 Suppl 6: vi89-98.
Kruit W H, Suciu S, Dreno B, et al. Selection of immunostimulant AS15 for active immunization with MAGE-A3 protein: results of a randomized phase II study of the European Organisation for Research and Treatment of Cancer Melanoma Group in Metastatic Melanoma. J Clin Oncol 2013; 31 (19): 2413-20.
Giraud M, Yoshida H, Abramson J, et al. Aire unleashes stalled RNA polymerase to induce ectopic gene expression in thymic epithelial cells. Proc Natl Acad Sci (S A 2012; 109 (2): 535-40.
Pinto D, Montani E, Bolli M, et al. A functional BCR in human IgA and IgM plasma cells. Blood 2013; 121 (20): 4110-4.
Klein L, Kyewski B, Allen P M, Hogquist K A. Positive and negative selection of the T cell repertoire: what thymocytes see (and don't see). Nat Rev Immunol 2014; 14 (6): 377-91.
Theobald M, Biggs J, Dittmer D, Levine A J, Sherman L A. Targeting p53 as a general tumor antigen. Proc Natl Acad Sci USA 1995; 92 (26): 11993-7.
Theobald M, Biggs J, Hernandez J, Lustgarten J, Labadie C, Sherman L A. Tolerance to p53 by A2.1-restricted cytotoxic T lymphocytes. J Exp Med 1997; 185 (5): 833-41.
Trager U, Sierro S, Djordjevic G, et al. The immune response to melanoma is limited by thymic selection of self-antigens. PLOS One 2012; 7 (4): e35005.
Dunn G P, Bruce A T, Ikeda H, Old L J, Schreiber R D. Cancer immunoediting: from immunosurveillance to tumor escape. Nat Immunol 2002; 3 (11): 991-8.
Dunn G P, Old L J, Schreiber R D. The immunobiology of cancer immunosurveillance and immunoediting. Immunity 2004; 21 (2): 137-48.
Schumacher T N, Schreiber R D. Neoantigens in cancer immunotherapy. Science 2015; 348 (6230): 69-74.
Gjertsen M K, Bakka A, Breivik J, et al. Ex vivo ras peptide vaccination in patients with advanced pancreatic cancer: results of a phase I/II study. Int.J Cancer 1996; 65 (4): 450-3.
Gjertsen M K, Bakka A, Breivik J, et al. Vaccination with mutant ras peptides and induction of T-cell responsiveness in pancreatic carcinoma patients carrying the corresponding RAS mutation. Lancet 1995; 346 (8987): 1399-400.
Gjertsen M K, Buanes T, Rosseland A R, et al. Intradermal ras peptide vaccination with granulocyte-macrophage colony-stimulating factor as adjuvant: Clinical and immunological responses in patients with pancreatic adenocarcinoma. Int J Cancer 2001; 92 (3): 441-50.
Sampson J H, Archer G E, Mitchell D A, et al. An epidermal growth factor receptor variant III-targeted vaccine is safe and immunogenic in patients with glioblastoma multiforme. Mol Cancer Ther 2009; 8 (10): 2773-9.
Sampson J H, Heimberger A B, Archer G E, et al. Immunologic escape after prolonged progression-free survival with epidermal growth factor receptor variant III peptide vaccination in patients with newly diagnosed glioblastoma. J Clin Oncol 2010; 28 (31): 4722-9.
Carbone D P, Ciernik I F, Kelley M J, et al. Immunization with mutant p53- and K-ras-derived peptides in cancer patients: immune response and clinical outcome. J Clin Oncol 2005; 23 (22) : 5099-107.
Toubaji A, Achtar M, Provenzano M, et al. Pilot study of mutant ras peptide-based vaccine as an adjuvant treatment in pancreatic and colorectal cancers. Cancer Immunol Immunother 2008; 57 (9): 1413-20.
Weden S, Klemp M, Gladhaug I P, et al. Long-term follow-up of patients with resected pancreatic cancer following vaccination against mutant K-ras. Int J Cancer 2011; 128 (5): 1120-8.
Sahin U, Derhovanessian E, Miller M, et al. Personalized RNA mutanome vaccines mobilize poly-specific therapeutic immunity against cancer. Nature 2017; 547 (7662): 222-6.
Carreno B M, Magrini V, Becker-Hapak M, et al. Cancer immunotherapy. A dendritic cell vaccine increases the breadth and diversity of melanoma neoantigen-specific T cells. Science 2015; 348 (6236): 803-8.
Linette G P, Becker-Hapak M, Skidmore Z L, et al. Immunological ignorance is an enabling feature of the oligo-clonal T cell response to melanoma neoantigens. Proc Natl Acad Sci USA 2019; 116 (47): 23662-70.
Hilf N, Kuttruff-Coqui S, Frenzel K, et al. Actively personalized vaccination trial for newly diagnosed glioblastoma. Nature 2019; 565 (7738): 240-5.
Feltkamp M C, Smits H L, Vierboom M P, et al. Vaccination with cytotoxic T lymphocyte epitope-containing peptide protects against a tumor induced by human papillomavirus type 16-transformed cells. Eur J Immunol 1993; 23 (9): 2242-9.
Noguchi Y, Chen Y T, Old L J. A mouse mutant p53 product recognized by CD4⁺ and CD8⁺ T cells. Proc Natl Acad Sci USA 1994; 91 (8): 3171-5.
Mandelboim O, Vadai E, Fridkin M, et al. Regression of established murine carcinoma metastases following vaccination with tumour-associated antigen peptides. Nat Med 1995; 1 (11): 1179-83.
Schulz M, Zinkernagel R M, Hengartner H. Peptide-induced antiviral protection by cytotoxic T cells. Proc Natl Acad Sci USA 1991; 88 (3): 991-3.
Thery C, Amigorena S. The cell biology of antigen presentation in dendritic cells. Curr Opin Immuno/2001; 13 (1): 45-51.
Guermonprez P, Valladeau J, Zitvogel L, Thery C, Amigorena S. Antigen presentation and T cell stimulation by dendritic cells. Annu Rev Immunol 2002; 20:621-67.
Gnjatic S, Atanackovic D, Matsuo M, et al. Cross-presentation of HLA class I epitopes from exogenous NY-ESO-1 polypeptides by nonprofessional APCs. J Immunol 2003; 170 (3): 1191-6.
Toes R E, Blom R J, Offringa R, Kast W M, Melief C J. Enhanced tumor outgrowth after peptide vaccination. Functional deletion of tumor-specific CTL induced by peptide vaccination can lead to the inability to reject tumors. J Immunol 1996; 156 (10): 3911-8.
Bijker M S, van den Eeden S J, Franken K L, Melief C J, Offringa R, van der Burg SH. CD8⁺CTL priming by exact peptide epitopes in incomplete Freund's adjuvant induces a vanishing CTL response, whereas long peptides induce sustained CTL reactivity. J Immunol 2007; 179 (8): 5033-40.
Bijker M S, van den Eeden S J, Franken K L, Melief C J, van der Burg S H, Offringa R. Superior induction of anti-tumor CTL immunity by extended peptide vaccines involves prolonged, DC-focused antigen presentation. Eur J Immunol 2008; 38 (4): 1033-42.
Sabbatini P, Tsuji T, Ferran L, et al. Phase I trial of overlapping long peptides from a tumor self-antigen and poly-ICLC shows rapid induction of integrated immune response in ovarian cancer patients. Clin Cancer Res 2012; 18 (23): 6497-508.
Janssen E M, Droin N M, Lemmens E E, et al. CD4⁺ T-cell help controls CD8⁺ T-cell memory via TRAIL-mediated activation-induced cell death. Nature 2005; 434 (7029): 88-93.
Heimberger A B, Crotty L E, Archer G E, et al. Epidermal growth factor receptor VIII peptide vaccination is efficacious against established intracerebral tumors. Clin Cancer Res 2003; 9 (11): 4247-54.
Mavroudis D, Bolonakis I, Cornet S, et al. A phase I study of the optimized cryptic peptide TERT (572y) in patients with advanced malignancies. Oncology 2006; 70 (4): 306-14.
Kenter G G, Welters M J, Valentijn A R, et al. Phase I immunotherapeutic trial with long peptides spanning the E6 and E7 sequences of high-risk human papillomavirus 16 in end-stage cervical cancer patients shows low toxicity and robust immunogenicity. Clin Cancer Res 2008; 14 (1): 169-77.
Welters M J, Kenter G G, Piersma S J, et al. Induction of tumor-specific CD4⁺ and CD8⁺ T-cell immunity in cervical cancer patients by a human papillomavirus type 16 E6 and E7 long peptides vaccine. Clin Cancer Res 2008; 14 (1): 178-87.
Kenter G G, Welters M J, Valentijn A R, et al. Vaccination against HPV-16 oncoproteins for vulvar intraepithelial neoplasia. N Engl J Med 2009; 361 (19): 1838-47.
Ramanathan R K, Lee K M, McKolanis J, et al. Phase I study of a MUC1 vaccine composed of different doses of MUC1 peptide with SB-AS2 adjuvant in resected and locally advanced pancreatic cancer. Cancer Immunol Immunother 2005; 54 (3): 254-64.
Yamamoto K, Ueno T, Kawaoka T, et al. MUC1 peptide vaccination in patients with advanced pancreas or biliary tract cancer. Anticancer Res 2005; 25 (5): 3575-9.
Wada H, Isobe M, Kakimi K, et al. Vaccination with NY-ESO-1 overlapping peptides mixed with Picibanil OK-432 and montanide ISA-51 in patients with cancers expressing the NY-ESO-1 antigen. J Immunother 2014; 37 (2): 84-92.
Leffers N, Lambeck A J, Gooden M J, et al. Immunization with a P53 synthetic long peptide vaccine induces P53-specific immune responses in ovarian cancer patients, a phase II trial. Int J Cancer 2009; 125 (9): 2104-13.
Speetjens F M, Kuppen P J, Welters M J, et al. Induction of p53-specific immunity by a p53 synthetic long peptide vaccine in patients treated for metastatic colorectal cancer. Clin Cancer Res 2009; 15 (3): 1086-95.
Vermeij R, Leffers N, Hoogeboom B N, et al. Potentiation of a p53-SLP vaccine by cyclophosphamide in ovarian cancer: a single-arm phase II study. Int J Cancer 2012; 131 (5): E670-80
Zeestraten E C, Speetjens F M, Welters M J, et al. Addition of interferon-alpha to the p53-SLP (R) vaccine results in increased production of interferon-gamma in vaccinated colorectal cancer patients: a phase I/II clinical trial. Int J Cancer 2013; 132 (7): 1581-91.
Chi M, Dudek A Z. Vaccine therapy for metastatic melanoma: systematic review and meta-analysis of clinical trials. Melanoma Res 2011; 21 (3): 165-74.
Yoshida K, Noguchi M, Mine T, et al. Characteristics of severe adverse events after peptide vaccination for advanced cancer patients: Analysis of 500 cases. Oncol Rep 2011; 25 (1): 57-62.
Chianese-Bullock K A, Woodson E M, Tao H, et al. Autoimmune toxicities associated with the administration of antitumor vaccines and low-dose interleukin-2. J Immunother 2005; 28 (4): 412-9.
Karanikas V, Colau D, Baurain J F, et al. High frequency of cytolytic T lymphocytes directed against a tumor-specific mutated antigen detectable with HLA tetramers in the blood of a lung carcinoma patient with long survival. Cancer Res 2001; 61 (9): 3718-24.
Yu J S, Liu G, Ying H, Yong W H, Black KL, Wheeler CJ. Vaccination with tumor lysate-pulsed dendritic cells elicits antigen-specific, cytotoxic T-cells in patients with malignant glioma. Cancer Res 2004; 64 (14): 4973-9.
Kandalaft L E, Chiang C L, Tanyi J, et al. A Phase I vaccine trial using dendritic cells pulsed with autologous oxidized lysate for recurrent ovarian cancer. J Transl Med 2013; 11:149.
Kamigaki T, Kaneko T, Naitoh K, et al. Immunotherapy of autologous tumor lysate-loaded dendritic cell vaccines by a closed-flow electroporation system for solid tumors. Anticancer Res 2013; 33 (7): 2971-6.
Harris J R, Markl J. Keyhole limpet hemocyanin (KLH): a biomedical review. Micron 1999; 30 (6): 597-623.
Lammers R J, Witjes W P, Janzing-Pastors M H, Caris C T, Witjes J A. Intracutaneous and intravesical immunotherapy with keyhole limpet hemocyanin compared with intravesical mitomycin in patients with non-muscle-invasive bladder cancer: results from a prospective randomized phase III trial. J Clin Oncol 2012; 30 (18): 2273-9.
Elsamadicy A A, Chongsathidkiet P, Desai R, et al. Prospect of rindopepimut in the treatment of glioblastoma. Expert Opin Biol Ther 2017; 17 (4): 507-13.
Sampson J H, Aldape K D, Archer G E, et al. Greater chemotherapy-induced lymphopenia enhances tumor-specific immune responses that eliminate EGFRvIII-expressing tumor cells in patients with glioblastoma. Neuro Oncol 2011; 13 (3): 324-33.
Schuster J, Lai R K, Recht L D, et al. A phase II, multicenter trial of rindopepimut (CDX-110) in newly diagnosed glioblastoma: the ACT III study. Neuro Oncol 2015; 17 (6): 854-61.
Takeuchi O, Akira S. Pattern recognition receptors and inflammation. Cell 2010; 140 (6): 805-20.
Pandey S, Kawai T, Akira S. Microbial sensing by Toll-like receptors and intracellular nucleic acid sensors. Cold Spring Harb Perspect Biol 2014; 7 (1): a016246.
Salaun B, Romero P, Lebecque S. Toll-like receptors' two-edged sword: when immunity meets apoptosis. Eur J Immunol 2007; 37 (12): 3311-8.
Urban-Wojciuk Z, Khan M M, Oyler B L, et al. The Role of TLRs in Anti-cancer Immunity and Tumor Rejection. Front Immunol 2019; 10:2388.
Saxena M, Bhardwaj N. Turbocharging vaccines: emerging adjuvants for dendritic cell based therapeutic cancer vaccines. Curr Opin Immunol 2017; 47:35-43.
Pichlmair A, Reis e Sousa C. Innate recognition of viruses. Immunity 2007; 27 (3): 370-83.
McCluskie M J, Krieg AM. Enhancement of infectious disease vaccines through TLR9-dependent recognition of CpG DNA. Curr Top Microbiol Immunol 2006; 311:155-78.
Speiser D E, Lienard D, Rufer N, et al. Rapid and strong human CD8⁺ T cell responses to vaccination with peptide, IFA, and CpG oligodeoxynucleotide 7909. J Clin Invest 2005; 115 (3): 739-46.
Appay V, Jandus C, Voelter V, et al. New generation vaccine induces effective melanoma-specific CD8⁺ T cells in the circulation but not in the tumor site. J Immunol 2006; 177 (3): 1670-8.
Pavlick A, Blazquez A B, Meseck M, et al. Combined Vaccination with NY-ESO-1 Protein, Poly-ICLC, and Montanide Improves Humoral and Cellular Immune Responses in Patients with High-Risk Melanoma. Cancer Immunol Res 2020; 8 (1): 70-80.
Zuckerman J N. The importance of injecting vaccines into muscle. Different patients need different needle sizes. BMJ 2000; 321 (7271): 1237-8.
Liang F, Lore K. Local innate immune responses in the vaccine adjuvant-injected muscle. Clin Transl Immunology 2016; 5 (4): e74.
Jennison C, Turnbull B W. Group sequential methods with applications to clinical trials. Boca Raton: Chapman and Hall/CRC; 2000.
Ivanova A, Qaqish B F, Schell M J. Continuous toxicity monitoring in phase II trials in oncology. Biometrics 2005; 61 (2): 540-5.
Gnjatic S, Ritter E, Buchler M W, et al. Seromic profiling of ovarian and pancreatic cancer. Proc Natl Acad Sci USA 2010; 107 (11): 5088-93.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A vaccine composition comprising at least two mutant-calreticulin (CALR)-peptides, wherein the at least two peptides have overlapping sequences.

2. The vaccine composition of claim 1, wherein the vaccine composition comprises at least three mutant-calreticulin (CALR)-peptides, alternatively at least four mutant-calreticulin (CALR)-peptides, alternatively at least five mutant-calreticulin (CALR)-peptides, wherein all peptides in the composition have overlapping sequences.

3. The vaccine composition of claim 1 or claim 2, wherein the overlapping sequences overlap by about 1 amino acid, alternatively by about 2 amino acids, alternatively by about 3 amino acids, alternatively by about 4 amino acids, alternatively by about 5 amino acids, alternatively by about 6 amino acids, alternatively by about 7 amino acids, alternatively by about 8 amino acids, alternatively by about 9 amino acids, alternatively by about 10 amino acids, alternatively by about 15 amino acids, alternatively by about 20 amino acids.

4. The vaccine composition of any one of the preceding claims, wherein the at least two peptides comprise at least about 20 amino acids, alternatively at least about 22 amino acids, alternatively at least about 24 amino acids, alternatively at least about 25 amino acids, alternatively at least about 27 amino acids.

5. A vaccine composition comprising at least two mutant-calreticulin (CALR)-peptides selected from the group consisting of SEQ ID NOs: 2, 3, 4, 5, and 6.

6. The vaccine composition of claim 5, wherein the vaccine composition comprises at least three mutant-calreticulin (CALR)-peptides, alternatively at least four mutant-calreticulin (CALR)-peptides, alternatively at least five mutant-calreticulin (CALR)-peptides selected from the group consisting of SEQ ID NOs: 2, 3, 4, 5, and 6.

7. The vaccine composition of any one of the preceding claims, wherein the vaccine composition further comprises a pharmaceutically acceptable carrier, an adjuvant, or helper peptide.

8. The vaccine composition of claim 7, wherein the adjuvant is selected from the group consisting of Polyinosinic-Polycytidylic Acid stabilized with Polylysine and Carboxymethylcellulose (Poly-ICLC), Keyhole limpet hemocyanin (KLH), and combinations thereof.

9. The vaccine composition of any one of the preceding claims, wherein the vaccine composition is configured to be administered intramuscularly, intranodally, subcutaneously, or intravenously.

10. The vaccine composition of any one of the preceding claims, wherein the vaccine composition induces or elicits an antitumor response or improves or enhances antitumor T cell immunity in a subject in need thereof.

11. The vaccine composition of claim 10, wherein the antitumor response is a CD4 and/or CD8 T cell response.

12. The vaccine composition of claim 10 or claim 11, wherein the subject is a human suffering from a hematological malignancy with a calreticulin mutation.

13. The vaccine composition of claim 12, wherein the hematological malignancy is a myeloproliferative neoplasm.

14. A method of inducing or eliciting an antitumor response or improving or enhancing antitumor T cell immunity in a subject in need thereof, the method comprising administering an effective amount of a vaccine composition of any one of claims 1-13.

15. The method of claim 14, wherein the subject is a human suffering from a hematological malignancy with a calreticulin mutation.

16. The method of claim 15, wherein the hematological malignancy is a myeloproliferative neoplasm.

17. A method of preventing, treating, reducing, or slowing progression or development of a hematological malignancy with a calreticulin mutation in a subject in need thereof, the method comprising administering an effective amount of a vaccine composition of any one of claims 1-13.

18. The method of claim 17, wherein the hematological malignancy is a myeloproliferative neoplasm.

19. A method of preventing, reducing, or slowing progression or development of a hematological malignancy with a calreticulin mutation in a subject at risk of developing a hematological malignancy with a calreticulin mutation, the method comprising determining if a subject is at risk of developing a hematological malignancy with a calreticulin mutation and administering to the subject an effective amount of a vaccine composition of any one of claims 1-13.

20. The method of claim 19, wherein determining if a subject is at risk of developing a hematological malignancy with a calreticulin mutation comprises detecting a calreticulin mutation in a sample from the subject.

21. The method of claim 20, wherein the sample is a biological sample.

22. The method of claim 21, wherein the biological sample is selected from the group consisting of blood, tissue, cells, urine, saliva, and biological fluids.

23. The method of any one of claims 19-22, wherein the hematological malignancy is a myeloproliferative neoplasm.
